(19) United States
(12) Patent Application Publication
(10) Pub. No.: US 2020/0360034 A1
(43) Pub. Date: Nov. 19, 2020

(54) Ear-Nose-Throat (ENT) Probe Tip Design to Facilitate Balloon Passage

(71) Applicant: Biosense Webster (Israel) Ltd., Yokneam (IL)

(72) Inventors: Yehuda Algawi, Binyamina (IL); Assaf Govari, Haifa (IL); Ilya Sitnitsky, Nahariya (IL)

(21) Appl. No.: 16/412,173

(22) Filed: May 14, 2019

(51) Int. Cl. A61B 17/24; A61B 1/233; A61M 25/00; A61M 25/10

(52) U.S. Cl. CPC A61B 17/24; A61B 1/233; A61M 29/02; A61M 25/10; A61M 25/0041

(57) ABSTRACT

A medical probe includes a shaft and a distal-end assembly. The shaft is configured for insertion into a cavity of an organ of a patient. The distal-end assembly is fitted at a distal end of the shaft and includes (a) a camera, which is fitted at a distal edge of the distal-end assembly and is configured to acquire images of a target tissue site in the cavity, (b) an expandable balloon, and (c) a recessed opening at the distal edge of the distal-end assembly for deploying and retracting the balloon. At least part of the recessed opening is slanted at an oblique angle relative to a longitudinal axis of the distal-end assembly, thereby causing the balloon to exit and expand while not obstructing the images acquired by the camera.

### FIELD OF THE INVENTION

The present invention relates generally to invasive medical probes, and particularly to probes for ear-nose-throat (ENT) applications.

### BACKGROUND OF THE INVENTION

Various types of invasive medical probes include mechanical designs intended to improve probe deployment at treated tissue. For example, U.S. Patent Application Publication 2009/0062871 describes balloon cannula systems that may be used for accessing and visualizing the spine, and related methods of treatment, including a forward-looking balloon system for creating a working space. In some embodiments, the balloon system has atraumatic dissection capability to allow visualization in spine. The devices and methods described may be used, for example, to perform denervation of neurological tissue, to dispense pharmacological agents, and to diagnose and treat spine disc related medical conditions.

As another example, U.S. Patent Application Publication 2012/0209273 describes systems and methods for minimally invasive discectomy procedures. In some variations, a tissue removal system may comprise a handheld housing, an outer shaft comprising a distal portion and a proximal portion coupled to the handheld housing, a distal sheath coupled to the distal portion of the outer shaft, a motor, an inner shaft coupled to the motor, where the inner shaft is located partially within the outer shaft and partially within the distal sheath, a tip portion coupled to a distal portion of the inner shaft, and an elongate member distally extending through a distal opening of the inner shaft, the elongate member having a retracted configuration and an extended configuration, where the distal sheath comprises at least one element (e.g. at least one protrusion) that engages the tip portion to couple the tip portion to the distal sheath.

### SUMMARY OF THE INVENTION

An embodiment of the present invention provides a medical probe including a shaft and a distal-end assembly. The shaft is configured for insertion into a cavity of an organ of a patient. The distal-end assembly is fitted at a distal end of the shaft and includes (a) a camera, which is fitted at a distal edge of the distal-end assembly and is configured to acquire images of a target tissue site in the cavity, (b) an expandable balloon, and (c) a recessed opening at the distal edge of the distal-end assembly for deploying and retracting the balloon. At least part of the recessed opening is slanted at an oblique angle relative to a longitudinal axis of the distal-end assembly, thereby causing the balloon to exit and expand while not obstructing the images acquired by the camera.

In some embodiments, the distal-end assembly further includes one or more LEDs configured to illuminate the cavity through the distal edge. In other embodiments, the distal-end assembly further includes one or more optical fibers configured to illuminate the cavity through the distal edge.

In some embodiments, the recessed opening is configured to permit passage of the expandable balloon when the balloon is collapsed.

There is additionally provided, in accordance with an embodiment of the present invention, a method, including inserting into a cavity of an organ of a patient a medical probe including a distal-end assembly which is fitted at a distal end of a shaft. The distal-end assembly includes: (a) a camera, which is fitted at a distal edge of the distal-end assembly and is configured to acquire images of a target tissue site in the cavity, (b) an expandable balloon, and (c) a recessed opening at the distal edge of the distal-end assembly for deploying and retracting the balloon. At least part of the recessed opening is slanted at an oblique angle relative to a longitudinal axis of the distal-end assembly, thereby causing the balloon to exit and expand while not obstructing the images acquired by the camera. The collapsed balloon is advanced through the recessed opening. The balloon is expanded.

In some embodiments, the method further includes passing the balloon in a collapsed configuration via the recessed opening, and then expanding the balloon.

The present invention will be more fully understood from the following detailed description of the embodiments thereof, taken together with the drawings in which:

### BRIEF DESCRIPTION OF THE DRAWINGS

FIG. 1 is a schematic, pictorial illustration of an ear-nose-throat (ENT) probe-based treatment system comprising an expandable balloon, in accordance with an embodiment of the present invention;

FIG. 2 is a schematic isometric view of a distal end of the ENT probe of FIG. 1, in accordance with an embodiment of the present invention; and

FIG. 3 is a flow chart that schematically illustrates a method for ENT balloon dilation treatment, in accordance with an embodiment of the present invention.

### DETAILED DESCRIPTION OF EMBODIMENTS

OVERVIEW

A very narrow channel in a patient's ear-nose-throat (ENT) system may cause, or worsen, an ENT medical condition. For instance, a Eustachian tube may be too narrow, and/or may be blocked by wax, which may cause repeated ear infections. As another example, the sinuses also include very narrow channels that may be blocked. These, and other related ENT medical conditions, may be relieved by dilating the channel.

Embodiments of the present invention that are described hereinafter provide improved probes and associated methods for balloon treatments, such as ENT balloon sinuplasty. The disclosed probes have a recessed opening through which an expandable balloon can be advanced beyond the probe and retracted back into the probe. The disclosed recessed opening is configured to prevent the balloon from being trapped or blocked at the distal end exit. Thus, the disclosed recess permits passage of a dilating balloon, while maintaining the outside diameter of the probe sufficiently small to enable access of the probe to narrow channels.

In some embodiments, a medical probe is provided, which comprises a shaft for insertion into a cavity of an organ of a patient, such as into the sinuses. The medical probe further comprises a distal-end assembly, which is fitted at a distal end of the shaft and comprises a camera, an expandable balloon, and a recessed opening at the distal edge of the distal-end assembly. The camera, which is fitted at the distal edge of the distal-end assembly, is configured to acquire images of a target tissue site in the cavity. The recessed opening at the distal edge of the distal-end assembly is configured for deploying and retracting the balloon. At least part of the recessed opening is slanted at an oblique angle relative to a longitudinal axis of the distal-end assembly, to cause the balloon to exit and expand while not obstructing the images acquired by the camera.

In some embodiments, the distal-end assembly comprises additional devices, such as one or more illuminating LEDs and/or fibers. The one or more LEDs and/or optical fibers are configured to illuminate the cavity through the distal edge (i.e., through the recessed opening).

The disclosed probe, which is capable of imaging and accessing narrow channels with a camera and an expandable balloon, respectively, by using a recessed opening of the distal edge of the probe, may allow the deployment of balloons that are otherwise too large to use, and thereby improve the clinical outcome of an ENT balloon characterization procedure.

SYSTEM DESCRIPTION

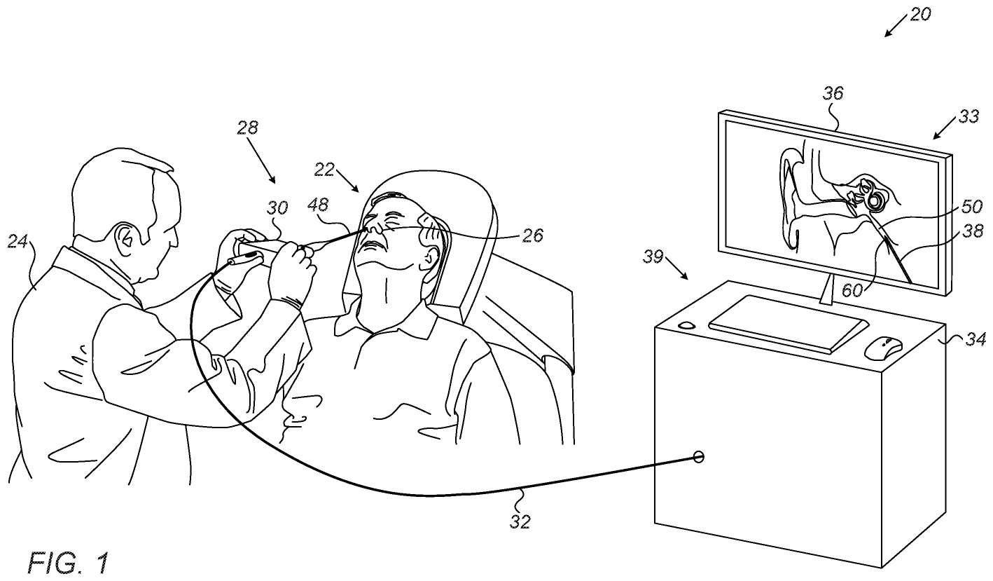

FIG. 1 is a schematic, pictorial illustration of an ear-nose-throat (ENT) probe-based treatment system comprising an expandable balloon 60, in accordance with an embodiment of the present invention. In the shown embodiment, expandable balloon 60 (seen on display 36) is fitted at a distal end 38 of a shaft 48 an ENT probe 28, which is configured to diagnose and/or treat an ENT medical condition, such as an infection in a Eustachian tube 50 of a patient 22.

A physician 24 inserts ENT probe 28 into a nose 26 of patient 22. ENT probe 28 comprises a handheld ENT apparatus 30, coupled to a proximal end of ENT probe 28 and configured to assist physician 24 to manipulate distal end 38 into Eustachian tube 50 through a nose 26 and in applying the balloon treatment.

In some embodiments, system 20 comprises console 33, which itself comprises a processor 34, typically a general-purpose computer, with suitable front end and interface circuits for receiving signals, such video images, via a cable 32 from probe tool 28 having a camera (not shown) mounted on probe tool 28.

Console 33 further comprises input devices 39 and a user display 36, which is configured to display relevant data (e.g., video images) received from processor 34 or inputs inserted by physician 24.

FIG. 1 shows only elements related to the disclosed techniques, for the sake of simplicity and clarity. System 20 typically comprises additional modules and elements that are not directly related to the disclosed techniques, such as probe position tracking, and are thus intentionally omitted from FIG. 1 and from the corresponding description.

Processor 34 may be programmed in software to carry out the functions that are used by the system, and to store data in a memory (not shown) to be processed or otherwise used by the software. The software may be downloaded to the processor in electronic form, over a network or, for example, may be provided on non-transitory electronic memory media. In particular, processor 34 runs a dedicated algorithm that enables processor 34 to perform the disclosed steps.

EAR-NOSE-THROAT (ENT) PROBE TIP DESIGN TO FACILITATE BALLOON PASSAGE

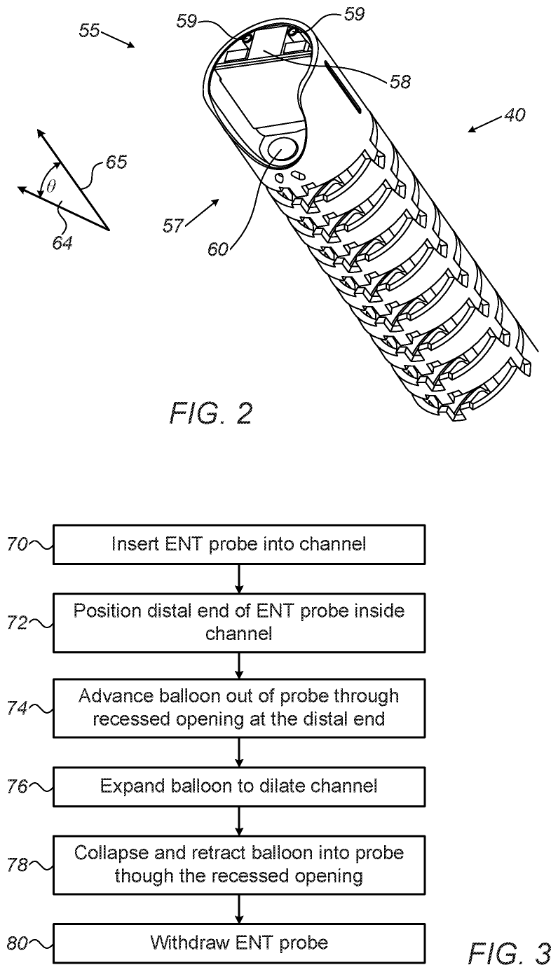

FIG. 2 is a schematic isometric view of distal end 38 of ENT probe 28 of FIG. 1, in accordance with an embodiment of the present invention. A distal-end assembly 40, which is fitted at distal end 38, comprises an opening 55 at the distal edge of distal-end assembly 40, through which expandable balloon 60 is advanced. As seen, opening 55 is partially blocked at its top with other devices, comprising, in the shown embodiment, a video camera 58 and illuminating LEDs and/or fiber optics, and irrigation tubes 59. Expandable balloon 60 is collapsed and retracted into distal-end assembly 40. Opening 55 comprises a rounded recess 57 at the bottom of opening 55, which enables distal-end assembly 40 to possess a sufficiently large aperture to allow advancement and retraction of collapsed balloon 60 through opening 55, while maintaining the diameter of distal-end assembly 40 sufficiently small to be able to provide access to, and to operate within, narrow ENT channels, such as the Eustachian tube. Rounded recess 57 at the bottom of the opening further enables an oblique passage (i.e., in a downward tilt at an angle θ (64) relative to a longitudinal axis 65 of the distal end) of expandable balloon 60 and when the balloon is expanded, to maintain the balloon tilted away that way from camera 58 so as not to obstruct the images of the target tissue site.

The example illustration shown in FIG. 2 is chosen purely for the sake of conceptual clarity. Only elements relevant to embodiments of the invention are shown. For example, in another embodiment, distal end 38 may additionally include a position sensor.

The example flow chart shown in FIG. 3 is chosen purely for the sake of conceptual clarity. Additional steps, such as fitting additional devices and mechanical controls to distal end 38, are omitted for simplicity of presentation.

FIG. 3 is a flow chart that schematically illustrates a method for ENT balloon dilation treatment, in accordance with an embodiment of the present invention. The process begins with physician 24 inserting ENT probe 28 into a narrow channel, such as eustachian tube 50, at an ENT probe insertion step 70. Next, physician 24 operates a video camera in probe 28, to position distal end 38 at a location inside the channel that requires balloon dilation, at an ENT probe positioning step 72.

Subsequently, physician 24 distally advances expandable balloon 60 through opening 55 comprising recess 57, at a balloon deployment step 74. Next, physician 24 expands balloon 60 to dilate eustachian tube 50, at a balloon expansion step 76. After performing the dilation, physician 24 operates probe 28 to collapse balloon 60 and retract the balloon back into probe 28 via opening 55 comprising recess 57, at a balloon retraction step 78. Finally, at a probe withdrawing step 80, physician 24 removes probe 28 from patient 22.

The example flow chart shown in FIG. 3 is chosen purely for the sake of conceptual clarity. In alternative embodiments, for example, based on indication of a position probe 28 from a position tracking system that tracks a position sensor in distal end 38, physician 54 may choose to change the location of distal end 38 inside eustachian tube 50.

Although the embodiments described herein mainly address ENT applications, the methods and systems described herein can also be used in other applications, such as in cerebrovascular applications.

It will be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A medical probe, comprising:
a shaft for insertion into a cavity of an organ of a patient; and
a distal-end assembly, which is fitted at a distal end of the shaft and comprises:
a camera, which is fitted at a distal edge of the distal-end assembly and is configured to acquire images of a target tissue site in the cavity;
an expandable balloon; and
a recessed opening at the distal edge of the distal-end assembly for deploying and retracting the balloon, wherein at least part of the recessed opening is slanted at an oblique angle relative to a longitudinal axis of the distal-end assembly, thereby causing the balloon to exit and expand while not obstructing the images acquired by the camera.

2. The medical probe according to claim 1, wherein the distal-end assembly further comprises one or more LEDs configured to illuminate the cavity through the distal edge.

3. The medical probe according to claim 1, wherein the distal-end assembly further comprises one or more optical fibers configured to illuminate the cavity through the distal edge.

4. The medical probe according to claim 1, wherein the recessed opening is configured to permit passage of the expandable balloon when the balloon is collapsed.

5. A method, comprising:
inserting into a cavity of an organ of a patient a medical probe comprising a distal-end assembly which is fitted at a distal end of a shaft, the distal-end assembly comprising:
a camera, which is fitted at a distal edge of the distal-end assembly and is configured to acquire images of a target tissue site in the cavity;
an expandable balloon; and
a recessed opening at the distal edge of the distal-end assembly for deploying and retracting the balloon, wherein at least part of the recessed opening is slanted at an oblique angle relative to a longitudinal axis of the distal-end assembly, thereby causing the balloon to exit and expand while not obstructing the images acquired by the camera;
advancing the collapsed balloon through the recessed opening; and
expanding the balloon.

6. The method according to claim 5, further comprising illuminating the cavity through the distal edge using one or more LEDs in the distal-end assembly.

7. The method according to claim 5, further comprising illuminating the cavity through the distal edge using one of one or more optical fibers in the distal-end assembly.

8. The method according to claim 5, wherein expanding the balloon comprises passing the balloon in a collapsed configuration via the recessed opening, and then expanding the balloon.
